(19) United States
(12) Patent Application Publication
(10) Pub. No.: US 2009/0036672 A1
(43) Pub. Date: Feb. 5, 2009

(54) INTERMEDIATE CEFDINIR SALTS

(75) Inventors: Giovanni Pozzi, Besana Brianza (IT); Patricio Martin Gomez, Salamanca (ES); Marco Alpegiani, Milano (IT); Walter Cabri, Rozzano (IT)

Correspondence Address: ROTHWELL, FIGG, ERNST & MANBECK, P.C., 1425 K STREET, N.W., SUITE 800, WASHINGTON, DC, 20005, US

(73) Assignee: ANTIBIOTICOS S.p.A., Rodano (IT)

(21) Appl. No.: 12/181,438

(22) Filed: Jul. 29, 2008

Related U.S. Application Data
(63) Continuation-in-part of application No. 10/529,649, filed on Oct. 11, 2005, now Pat. No. 7,405,294.

(30) Foreign Application Priority Data
Oct. 1, 2002 (IT) MI2002A002076

(51) Int. Cl. C07D 501/04; C07D 501/16

(52) U.S. Cl. 540/222

(57) ABSTRACT

Disclosed are salts of the general formula (I)

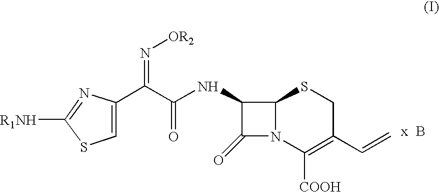

wherein R₁, R₂ and B are as defined in the description and methods for the preparation thereof.

These salts are useful intermediates for the preparation of cefdinir.

### FIELD OF THE INVENTION

The present invention relates to cephalosporins, in particular to cefdinir intermediates and to a process for the preparation of said intermediates.

### SUMMARY

The present invention relates to salts of general formula (I)

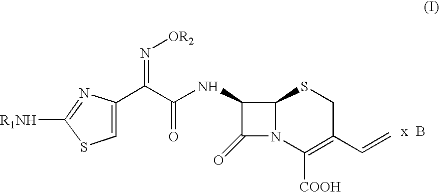

wherein R₁, R₂ and B are as defined in the description. These salts are useful as intermediates for the preparation of cefdinir.

Compounds (I) can be obtained through a process comprising the reaction of a compound of formula (II)

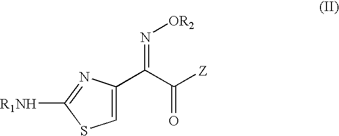

wherein R₁ and R₂ are as defined in the description, with 7-amino-3-vinyl-3-cephem-4-carboxylic acid of formula (III)

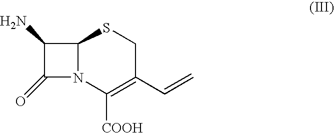

### BACKGROUND OF THE INVENTION

[(−)-(6R,7R)]-7-((Z)-2-(2-Amino-4-thiazol)-2-hydroxyiminoacetamido)-8-oxo-3-vinyl-5-thia-1-azabicyclo[4.2.0]-oct-2-ene-2-carboxylic acid (IV), commonly known as cefdinir,

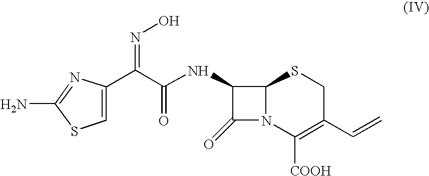

is a third generation semisynthetic cephalosporin for oral use, characterized by a broad antibacterial spectrum against gram-positive and gram-negative bacteria; its antibiotic activity is higher than that of other antibiotics for oral administration. In particular, it shows excellent antibacterial activity against staphylococci and streptococci.

Cefdinir is usually synthesized through intermediates of formula (V) wherein the hydroxyimino group (and optionally the primary amino group) is protected

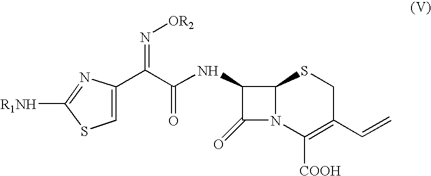

wherein R₁ and R₂ are as defined in the description.

According to the literature, the intermediates (V) can be obtained in different ways, but their recovery is troublesome and not convenient from the industrial standpoint.

For example, according to WO 97/24358, an intermediate of formula (V) wherein R₁ is hydrogen and R₂ is trityl (Va), is recovered as the salt with p-toluenesulfonic acid (VIa)

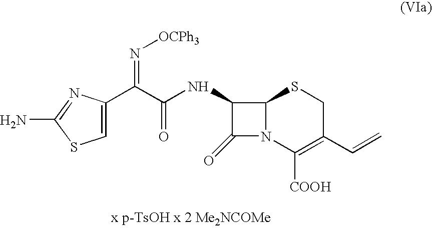

The drawback of this method is that the recovery is accomplished by adding to the reaction mixture anti-solvents such as ethers, which are dangerous and therefore not suitable for industrial use.

Other methods do not envisage recovery of the intermediates (V); as a consequence, the quality of the final product is poor and further purifications are required (WO 98/45299; Kamachi, H. et al., J. Antibiot. 1988 41(11), 1602-16).

Alternatively, the side chain can be linked to the cephalosporanic nucleus by means of subsequent synthetic steps, with decrease in the overall yield and increase in the process time (U.S. Pat. No. 4,559,334, EP 304019).

The intermediates (V) can also be recovered from water as free acids, but filtration and drying are very difficult.

### BRIEF DESCRIPTION OF THE DRAWINGS

FIG. 1 is the ¹³C-NMR spectrum obtained from the compound prepared in Example 5. NMR experiments were run on a Varian Mercury 300 MHz spectrometer. A product sample was dissolved in deuterated dimethylsulfoxide and the following parameters were applied in each sequence: pulse width 90°, delay 3s, transient number 1024, sweep width 0-220 ppm, probe temperature 25° C.

FIG. 2 is the DSC spectrum obtained from the compound prepared in Example 5. DSC was obtained at a heating rate of 11° C./min starting from 40° C. to 200° C. using Perkin Elmer Pyris 1 DSC equipment. The product showed a broad endotherm due to water and solvent loss. A melting endotherm followed by decomposition was detected at 172° C.

FIG. 3A is the XRPD spectrum obtained from the compound prepared in Example 5. The sample product was analyzed through Bragg geometry in the range from 0-40°2Θ in a continuous scan (1 s/sweep). A Diffractometer Philips Analytical X-Ray PW1800 equipped with a Cu tube anode was used.

FIG. 3B is a table of angles of some XRDP reflections (angle °2Θ) corresponding to the spectrum obtained from the compound prepared in Example 5. The * symbol indicates 100% relative intensity.

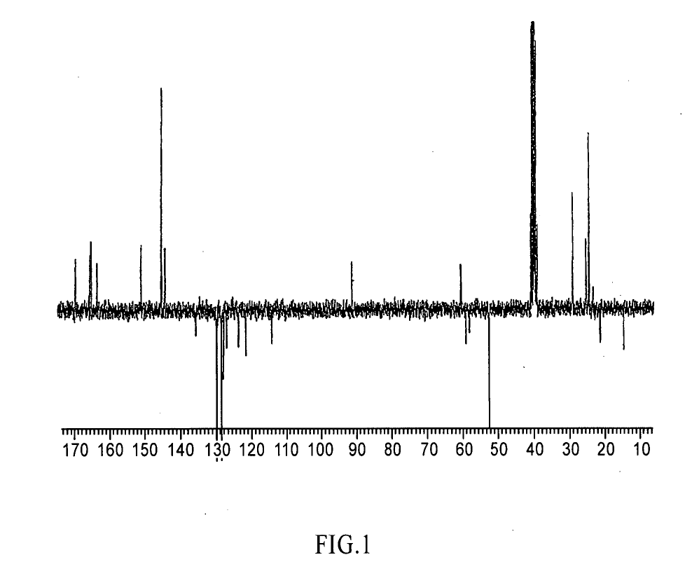

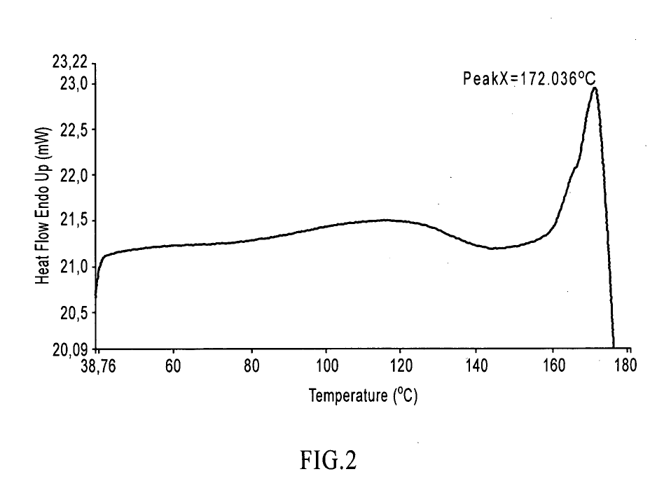

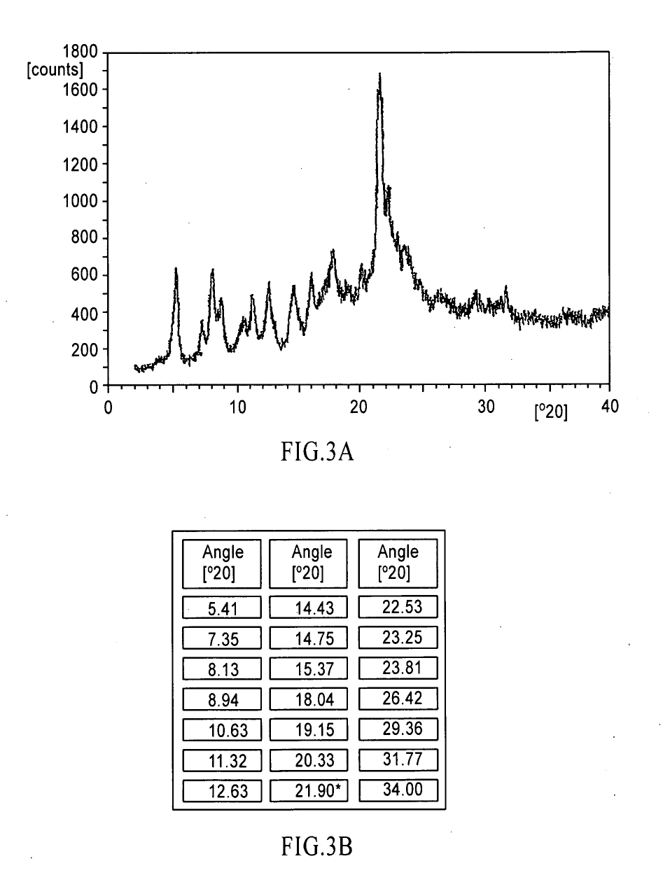

### DETAILED DESCRIPTION OF THE INVENTION

It has now been found that the intermediates (V) can be recovered in high yield and purity as the salts with ammonia or organic bases, in inert organic solvents of common industrial use, thus remarkably improving the manufacture of cefdinir in terms of time, costs and quality of the end product.

Accordingly, the present invention relates to salts of formula (I)

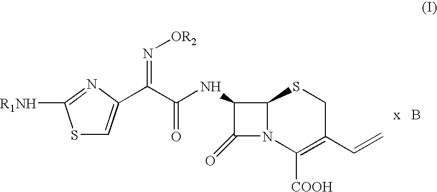

wherein
R₁ is hydrogen or an amino-protecting group, for example a C₁-C₆ acyl group, preferably formyl, wherein the acyl group is of the formula RCO— wherein R is H or a C₁-C₅ aliphatic group, preferably alkyl or alkenyl, or a C₁-C₅ alicyclic group, or wherein together with the carbonyl carbon to which it is attached, R forms a C₃-C₆ alicyclic or aromatic group, and wherein R is optionally substituted with one or more fluorine or chlorine atoms; an alkyl- or aryl-oxycarbonyl group, preferably tert-butoxycarbonyl or p-methoxybenzyloxycarbonyl; or a trityl group wherein each benzene ring is optionally substituted with one or more methoxy and/or methyl groups, preferably unsubstituted trityl;
R₂ is a hydroxy-protecting group, for example a straight or branched C₁-C₆ alkyl group, preferably tert-butyl; a benzyl, benzhydryl or trityl group, wherein each benzene ring is optionally substituted with one or more methoxy, nitro and/or methyl group, preferably p-methoxybenzyl, 3,4-dimethoxybenzyl, benzhydryl, bis(p-methoxyphenyl)methyl or unsubstituted trityl;
B is ammonia or an organic base which is primary amines, preferably cyclohexylamine, 2-ethylhexylamine, benzylamine, α-methylbenzylamine and tert-octylamine; secondary amines, preferably diethylamine, morpholine, dicyclohexylamine, N,N-methylbenzylamine or N,N′-dibenzylethylenediamine; tertiary amines, preferably triethylamine, tributylamine, triisooctylamine, ethyldiisopropylamine, N-methylmorpholine, pyridine, 2,6-lutidine or quinoline; guanidine, preferably 1,1,3,3-tetramethylguanidine; amidines, preferably 1,5-diazabicyclo[4.3.0]non-5-ene (DBN) or 1,8-diazabicyclo[5.4.0]undec-7-ene (DBU).

The present invention also relates to hydrates, solvates or adducts of the salts of formula (I), wherein said adduct is a complex of said salt and a compound represented as H-Z, wherein

H-Z is preferably a substituted or unsubstituted phenol or thiophenol or a substituted or unsubstituted hydroxyl- or mercapto-heterocycle; preferably Z is —Cl, —O—P⁺(Ph)₃Cl⁻, —O—P(S)(OEt)₂, —O—P(O)(OEt)₂, —O—SO₂Me, —O—SO₂Ph, —O—SO₂-pTol, —O—COtBu, —O—C(O)OEt, —O—CH═N⁺(CH₃)₂Cl— or benzotriazol-1-yl-3-oxide; more preferably Z is —S-mercaptobenzothiazolyl, —O-benzotriazol-1-yl, or —S-(2-methyl-thiadiazol-5-yl).

A preferred salt according to the invention is the dicyclohexylamine salt of the formula (Ia)

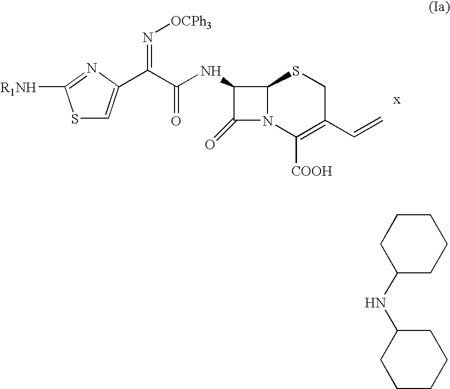

The salts of formula (I) are obtained through a process which envisages three possible alternatives, whose common feature is that the acids of the formula (V) are not isolated. The alternatives are illustrated in the following scheme.

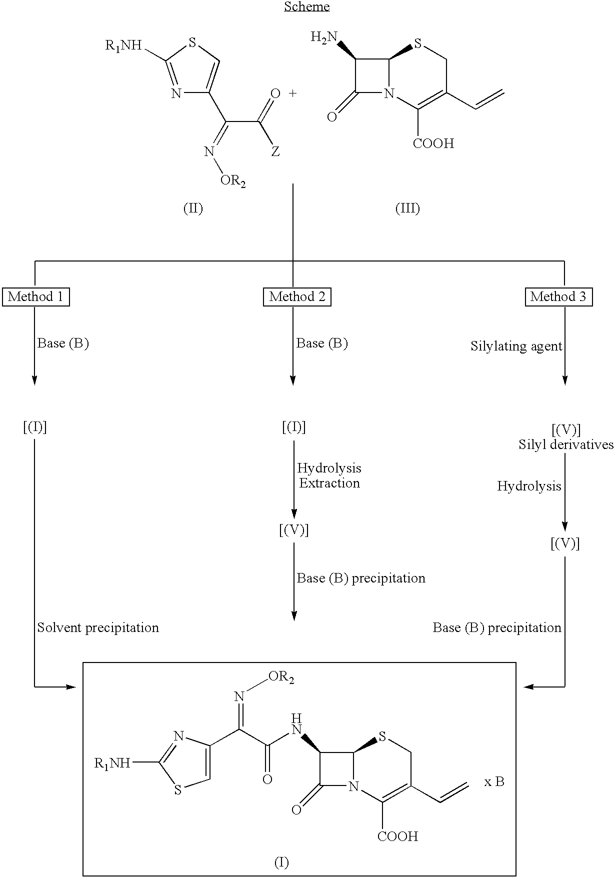

In a first embodiment of the invention (method 1), an activated 2-(aminothiazol-4-yl)-2-(hydroxyimino)acetic acid derivative of formula (II)

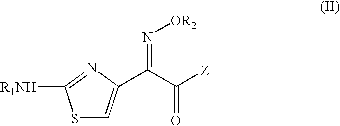

wherein R₁ and R₂ are as defined above and Z is a carboxy-activating group which, together with the adjacent carbonyl group, forms an acyl halide, an ester, a thioester, an anhydride, or a mixed anhydride with a carboxylic or sulphonic acid, or is a group of formula —O—P⁺(Ph)₃Cl⁻, —O—P(S)(OEt)₂, —O—P(O)(OEt)₂, —O—C(O)OEt, —O—CH═N⁺(CH₃)₂Cl⁻ or benzotriazol-1-yl-3-oxide. Preferably, Z is selected from —Cl, —S-mercaptobenzothiazolyl, —O—SO₂Me, —O—SO₂Ph, —O—SO₂-pTol, —O—COtBu, —O-benzotriazol-1-yl, —S-(2-methyl-thiadiazol-5-yl);

is reacted with 7-amino-3-vinyl-3-cephem-4-carboxylic acid (III),

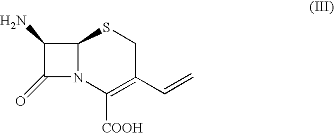

in the presence of ammonia or an organic base, B, described above. The compounds (II) and (III) comprise also their hydrates and solvates. The reaction is carried out in an organic solvent that forms a solution with the reactants, is inert to the reactants, and does not interfere with the reaction. The organic solvent is generally a: halogenated C₁-C₈ hydrocarbon, preferably methylene chloride; C₁-C₈ carboxylic acid ester, preferably dimethylcarbonate, ethyl acetate or butyl acetate; a ketone, preferably acetone, methyl ethyl ketone or methyl isobutyl ketone; a nitrile, preferably acetonitrile or propionitrile; an C₁-C₈ amide, preferably N,N-dimethylformamide, N,N-dimethylacetamide, or N-methylpyrrolidone; an aromatic hydrocarbon, preferably toluene or xylene; an C₁-C₈ ether, preferably tetrahydrofuran, dioxane or ethylene glycol dimethyl ether; a C₁-C₈ sulfoxide or sulfone, preferably dimethylsulfoxide, dimethyl sulfone or sulfolane; an C₁-C₈ alcohol, preferably methanol or ethanol, or mixtures thereof, optionally in the presence of water, at a temperature ranging from −20° C. to +80° C., preferably from 0° C. to 40° C. Preferred solvents according to the invention are N,N-dimethylformamide and N,N-dimethylacetamide. The amount of base can be stoichiometric to the compound of formula (III) or in molar excess up to 3 times, preferably ranging from 1 to 2 equivalents. The compound of formula (II) is used in an amount of 0.5 to 2.0 equivalents, preferably 0.8 to 1.2 equivalents or more preferably 0.9 to 1.1 equivalents, with respect to the compound of formula (III).

The resulting salts of the formula (I) precipitate by addition of an anti-solvent which is suitably selected to promote crystallization. The anti-solvent may be one of the solvents listed above, preferably diethylether, diisopropylether or ethylacetate. The anti-solvent is added to the reaction mixture in an amount of 1 to 6 times by volume with respect to the reaction solvent, preferably 2 to 3 times by volume. The crystallization temperature may range from −20° C. to 50° C., preferably from −10° C. to 30° C.

In a second embodiment of the invention (method 2) the reaction is carried out as described above, but the salts (I) are not immediately precipitated, but rather are converted to an acid of the formula (V), by addition of aqueous solvent, such as a solution of ethyl acetate and water, and adjustment of the solution to a pH ranging from 1.0-6.0, preferably 2.0-4.0, more preferably 2.8-3.2, by addition of acid, preferably a strong acid such as hydrochloric acid, sulfuric acid, or nitric acid. The acid of formula (V) is extracted from the reaction mixture and precipitated from the extraction solvent by treatment with ammonia or an amine selected from those listed above, which can be the same or different from that used in the previous step. The salt is precipitated using an amount of base stoichiometric to the acid of the formula (V) or in molar excess of up to two times, preferably ranging from 1 to 1.5 equivalents. Also in this case the crystallization temperature may range from −20° C. to 50° C., preferably from −10° C. to 30° C. According to a preferred embodiment of this method, compounds (II) and (III) are reacted with 1,1,3,3-tetramethylguanidine or triethylamine. Preferably, the compound of formula (II) is the S-mercaptobenzothiazolyl thioester (IIa)

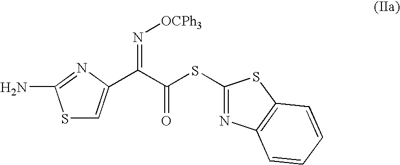

and the compound of formula (III) is 7-amino-3-vinyl-3-cephem-4-carboxylic acid (III)

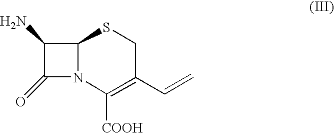

In a third embodiment of the invention (method 3), the reaction between the compounds (II) and (III) is carried out in the presence of a silylating agent, such as N-trimethylsilyacetamide, trimethylsilylchloride, hexamethyldisilazane, trimethylsilyldimethylamine, and preferably N,O-bis-trimethylsilylacetamide. The silylating agent is added in an amount of 0.5 to 2.0 molar equivalents with respect to compound (III), preferably 0.8 to 1.5 molar equivalents. The acid of formula (V) obtained after hydrolysis is extracted and precipitated as a salt of formula (I) by treatment with ammonia or with an amine selected from those listed above. Also in this case, use will be made of an amount of base stoichiometric to the acid of formula (V) or in molar excess up to two times, preferably ranging from 1 to 1.5 equivalents. According to a preferred embodiment of this method, the ester (IIa) is reacted with the acid (IIIa) in the presence of N,O-bis-trimethylsilylacetamide, to give, after hydrolysis, the acid (Va)

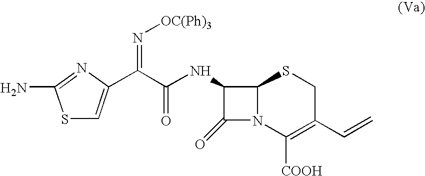

Among the three methods disclosed above, the second and the third ones are particularly preferred, as they allow one to obtain the salts of formula (I) with higher purity.

The reactions in the forgoing examples take place under ambient conditions unless otherwise specified, but the reaction temperatures may range from a temperature just above the freezing point of the solvent to a temperature just below the boiling point of the solvent. In an aqueous solution, for example, the temperature may range from about two degrees Celsius to about two-hundred degrees Celsius. Preferably, the temperature of the solution is selected such that it can be lowered to promote precipitation during the crystallization step.

The salts (I) precipitate as crystals from the reaction mixture and can be easily recovered by filtration or centrifugation. Through crystallization of the salts (I), the intermediates (V) are removed off the reaction medium; degradation is thus remarkably reduced, while the yield and quality of the intermediates are increased. The salts (I) can be obtained in the anhydrous form, or as hydrates, or can also be recovered as solvates. Hydration water or solvation solvent can be sometimes removed in part or almost completely by drying under reduced pressure, which increases the stability of the product. Typically, a salt having a water content of 0.5% or lower and a solvent content of 3% or lower can be obtained after drying. The salts of formula (I) can also be recovered as adducts with derivatives of formula H-Z wherein Z is as defined above. The derivatives of formula H-Z can be present in a molar ratio of 1:1 or lower with respect to the salt of formula (I). The salts of general formula (I) can be prepared by addition of a compound of formula H-Z to a solution of an acid of formula (V), followed by addition of a base of formula B.

The conversion of the salts (I) to cefdinir (IV) by removal of the protecting groups can be carried out according to methods already known in the literature (WO 0179211, WO 9724358, Kamachi, H. et al., J. Antibiot. 1988 41(11), 1602-16), the disclosures of which are hereby incorporated by reference.

The following examples illustrate the invention in greater detail.

### EXAMPLES

### Example 1

### Preparation of 7-[2-(aminothiazol-4-yl)-2-(trityloxyimino)-acetamido]-3-vinyl-3-cephem-4-carboxylic acid dicyclohexylamine salt

1,1,3,3-Tetramethylguanidine (35.8 ml) is added in 15 min to a suspension of 7-amino-3-vinyl-3-cephem-4-carboxylic acid (65.0 g) in N,N-dimethylformamide (0.78 L) previously cooled to 10° C. and the mixture is stirred at this temperature until complete dissolution. 2-(Aminothiazol-4-yl)-2-(trityloxyimino)acetic acid S-mercaptobenzothiazolic ester (172.7 g) is added thereto in 15 min and the mixture is stirred at this temperature until complete conversion of 7-amino-3-vinyl-3-cephem-4-carboxylic acid (HPLC analysis). After completion of the reaction, water (1.7 L) and ethyl acetate (2.2 L) are added and the pH is adjusted to 3.0 with diluted hydrochloric acid. The phases are separated and the organic one is washed with a 20% sodium chloride solution in water (0.86 L). The phases are separated and dicyclohexylamine (54.1 ml) is added in 30 min to the organic one. Formation of crystals is observed. After further 15 min the mixture is cooled to 0° C., stirred at this temperature for 1 hour, thereafter the precipitate is filtered, washed with ethyl acetate (1.7 L) and dried. 226.0 g of the title compound are obtained.

¹H-NMR (DMSO-d₆, 300 MHz): 9.86 (1H, d, J=8.3 Hz, —CONH—), 7.34-7.20 (15H, m, Ph₃), 7.01 (1H, dd, J=17.9 and 11.6 Hz, —CH═CH₂), 6.59 (1H, s, H-heteroaryl), 5.78 (1H, dd, J=8.3 and 5.0 Hz, —CONH—CH—), 5.24 (1H, d, J=17.9 Hz, —CH═CHH trans), 5.15 (1H, d, J=5.0 Hz, —CON—CH—), 5.00 (1H, d, J=11.6 Hz, —CH═CHH cis), 3.61 (1H, AB system, J_{AB}=17.0 Hz, —SCH₂), 3.46 (1H, AB system, J_{AB}=1.70 Hz, —SCH₂), 3.06-3.00 (2H, m, 2×HN—CH dicyclohexylamine), 1.99-1.06 (20H, m, 10×CH₂ dicyclohexylamine).

### Example 2

### Preparation of 7-[2-(aminothiazol-4-yl)-2-(trityloxyimino)-acetamido]-3-vinyl-3-cephem-4-carboxylic acid dicyclohexylamine salt

Triethylamine (9.1 ml) is added in 20 min to a suspension of 7-amino-3-vinyl-3-cephem-4-carboxylic acid (7.5 g) in N,N-dimethylformamide (90 ml) previously cooled to 15° C. 2-(Aminothiazol-4-yl)-2-(trityloxyimino)acetic acid S-mercaptobenzothiazolic ester (19.7 g) is added thereto in 15 min and the mixture is stirred at this temperature until complete conversion of 7-amino-3-vinyl-3-cephem-4-carboxylic acid (HPLC analysis). After completion of the reaction water (200 ml) and ethyl acetate (250 ml) are added and the pH is adjusted to 3.0 with diluted hydrochloric acid. The phases are separated and the organic one is washed with a 20% sodium chloride solution in water (200 ml). The phases are separated and dicyclohexylamine (7.2 ml) is added to the organic one in 15 min. Formation of crystals is observed. After further 15 min the mixture is cooled to 0° C., stirred at this temperature for 1 hour, thereafter the precipitate is filtered, washed with ethyl acetate (100 ml) and dried. 26.4 g of the title compound are obtained.

### Example 3

### Preparation of 7-[2-(aminothiazol-4-yl)-2-(trityloxyimino)-acetamido]-3-vinyl-3-cephem-4-carboxylic acid dicyclohexylamine salt

N,O-bistrimethylsilylacetamide (8.0 ml) is added in 15 min to a suspension of 7-amino-3-vinyl-3-cephem-4-carboxylic acid (7.5 g) in N,N-dimethylacetamide (50 ml) at 25° C. After further 20 min, 2-(aminothiazol-4-yl)-2-(trityloxyimino)acetic acid S-mercaptobenzothiazolic ester (19.8 g) is added and the mixture is stirred at this temperature until complete conversion of 7-amino-3-vinyl-3-cephem-4-carboxylic acid (HPLC analysis). After completion of the reaction, ethyl acetate (250 ml) and methanol (3 ml) are added and the mixture is stirred for 15 minutes. Water is then added (200 ml) and stirring is continued for further 15 min.

The phases are separated and the organic one is washed with a 20% sodium chloride solution in water (200 ml). The phases are separated and dicyclohexylamine (7.2 ml) is added to the organic phase in 15 min. Formation of crystals is observed. The mixture is allowed to stand for further 15 min, cooled to 0° C. and stirred at this temperature for 1 hour. The precipitate is filtered, washed with ethyl acetate (100 ml) and dried. 25.8 g of the title compound are obtained.

### Example 4

### Preparation of 7-[2-(aminothiazol-4-yl)-2-(trityloxyimino)-acetamido]-3-vinyl-3-cephem-4-carboxylic acid (R)-(+)-α-methylbenzylamine salt

The same procedure as example 3 is initially followed. After washing the organic phase with aqueous sodium chloride, (R)-(+)-amethylbenzylamine (4.6 ml) is added in 15 minutes. Formation of crystals is observed. The mixture is allowed to stand for further 15 min, cooled to 0° C. and stirred at this temperature for 1 hour. The precipitate is filtered, washed with ethyl acetate (100 ml) and dried. 20.4 g of the title compound are obtained.

¹H-NMR (DMSO-d₆, 300 MHz): 9.84 (1H, d, J=8.0 Hz, —CONH—), 7.49-7.18 (20H, m, 4×Ph), 7.01 (1H, dd, J=17.6 and 11.0 Hz, —CH═CH₂), 6.59 (1H, s, 1-1-heteroaryl), 5.77 (111, dd, J=8.0 and 5.0 Hz, —CONH—CH—), 5.20 (1H, d, J=17.6 Hz, —CH═CHH trans), 5.13 (1H, d, J=5.0 Hz, —CON—CH—), 4.97 (1H, d, J=111.6 Hz, —CH═CHH cis), 4.34 (1H, q, J=6.9 Hz, CHMe benzylamine), 3.58 (1H, AB system, J_{AB}=17.1 Hz, —SCH₂), 3.45 (1H, AB system, J_{AB}=17.1 Hz, —SCH₂), 1.47 (3H, d, J=6.9 Hz, Me).

### Example 5

### Preparation of (6R,7R)-,7-[[(2Z)-(2-amino-4-thiazolyl) [(triphenylmethoxy)imino]acetyl]amino]-3-ethenyl-8-oxo-5-thia-1-azabicyclo [4.2.0]oct-2-ene-2-carboxylic acid, N-dicyclohexylamine salt, adduct with 2-mercaptobenzothiazole (about 1:1:0.5)

A solution of 7-amino-3-vinyl-3-cephem-4-carboxylic acid (ca. 1.0 kg) and 1,1,3,3-tetramethylguanidine (ca. 0.5 kg) in N,N-dimethylformamide (ca. 11.5 kg) was treated with 2-(aminothiazol-4-yl)-2-(trityloxyimino)acetic acid S-mercaptobenzothiazolic ester (ca. 2.6 kg) and the mixture was stirred at <20° C. until complete conversion of 7-amino-3-vinyl-3-cephem-4-carboxylic acid (HPLC analysis). After completion of the reaction, water and ethyl acetate (50 L, ca. 1:1) were added and the pH was adjusted to about 3.0 with hydrochloric acid. The organic phase was separated and washed with a 20% aqueous sodium chloride (ca. 15 L). Upon addition of dicyclohexylamine (ca. 0.8 kg) to the organic phase, formation of a crystalline product was observed. The suspension was cooled, filtered and washed with ethyl acetate. After drying under vacuum, about 3.5 kg of the title compound was obtained.

¹H-NMR (DMSO-d₆, 300 MHz): 9.86 (1H, d, J=8.3 Hz, —CONH—), 7.56 (0.6H, d, J=7.2 Hz, —CH 2-mercaptobenzothiazole), 7.40-7.10 (19H, m, Tr, —NH₂, 2-mercaptobenzothiazole), 7.01 (1H, dd, J=17.6 and 11 Hz, —CH═CH₂), 6.60 (1H, s, H-heteroaryl), 5.78 (1H, dd, J=4.4 and 8.3, —CONH—CH—), 5.25 (1H, d, J=17.6 Hz, —CH═CHH trans), 5.15 (1H, d, J=4.4 Hz, —CON—CH—), 5.01 (1H, d, J=11.0 Hz, —CH═CHH cis), 4.01 (1.7H, q, J=7.2 Hz, CH₂-AcOEt), 3.61, 3.66 (2H, AB quartet, J_{AB}=17.1 Hz, —SCH₂), 3.04 (2.16H, m, HN—CH dicyclohexylamine), 2.87, 2.71 (0.06H, s, CH3 DMF), 2.2-1.0 (26H, m, CH₃-AcOEt and CH₂ dicyclohexylamine).

IR (KBr, cm⁻¹): 3022, 2933, 2856, 1782, 1692, 1581.

### Example 6

### Preparation of (6R,7R)-,7-[[(2Z)-(2-amino-4-thiazolyl) [(triphenylmethoxy)imino]acetyl]amino]-3-ethenyl-8-oxo-5-thia-1-azabicyclo[4.2.0]oct-2-ene-2-carboxylic acid, N-dicyclohexylamine salt, adduct with 2-mercaptobenzothiazole

2-Mercaptobenzothiazol (26.2 g) was added to a solution of (6R,7R)-,7-[[(2Z)-(2-amino-4-thiazolyl)[(triphenylmethoxy)imino]acetyl]amino]-3-ethenyl-8-oxo-5-thia-1-azabicyclo[4.2.0]oct-2-ene-2-carboxylic acid (100.0 g) in ethyl acetate (1.4 L) at room temperature and the resulting mixture was stirred at this temperature until complete dissolution. Dicyclohexylamine (28.4 g) was then added in 30 min to the solution. Formation of crystals was observed. After further 15 min the mixture was cooled to 0° C., stirred at this temperature for 1 hour, thereafter the precipitate was filtered, washed with ethyl acetate (1.1 L) and dried, affording 141.3 g of the title compound.

### Example 7

### Preparation of (6R,7R)-,7-[[(2Z)-(2-amino-4-thiazolyl)[(triphenylmethoxy)imino]acetyl]amino]-3-ethenyl-8-oxo-5-thia-1-azabicyclo[4.2.0]oct-2-ene-2-carboxylic acid, N-dicyclohexylamine salt, adduct with 2-mercaptobenzothiazole

2-Mercaptobenzothiazol (2.51 g) was added to a solution of (6R,7R)-,7-[[(2Z)-(2-amino-4-thiazolyl)[(triphenylmethoxy)imino]acetyl]amino]-3-ethenyl-8-oxo-5-thia-1-azabicyclo[4.2.0]oct-2-ene-2-carboxylic acid (16.0 g) in ethyl acetate (220 mL) at room temperature and the mixture was stirred at this temperature until complete dissolution. Dicyclohexylamine (4.54 g) was added in 30 min to the solution and formation of crystals was observed. After further 15 min the mixture was cooled to 0° C., stirred at this temperature for 1 hour, thereafter the precipitate was filtered, washed with ethyl acetate (170 mL) and dried, yielding 22.3 g of the title compound.

1. A salt of formula (I) 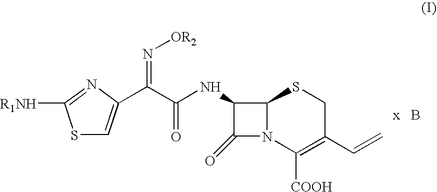
wherein
R₁ is hydrogen or an amino-protecting group which is a C₁-C₆ acyl group optionally substituted with one or more chlorine or fluorine atoms, an alkyl- or aryl-oxycarbonyl group, or a trityl group wherein each benzene ring is optionally substituted with one or more methoxy and/or methyl groups;
R₂ is a hydroxy-protecting group which is a straight or branched C₁-C₆ alkyl group, a benzyl, benzhydryl or trityl group wherein each benzene ring is optionally substituted with one or more methoxy, nitro and/or methyl groups;
B is ammonia or an organic base which is a primary amine, a secondary amine, a tertiary amine, guanidine, a guanidine derivative, or an amidine;
or a hydrate, solvate or adduct thereof.

2. A salt as claimed in claim 1 wherein R₁ is a formyl, tert-butoxycarbonyl, p-methoxybenzyloxycarbonyl, or trityl group.

3. A salt as claimed in claim 1 wherein R₂ is a tert-butyl, p-methoxybenzyl, 3,4-dimethoxybenzyl, benzhydryl, bis(p-methoxyphenyl)methyl or trityl group.

4. A salt as claimed in claim 1 wherein R₁ is hydrogen and R₂ is trityl.

5. A salt as claimed in claim 1 wherein B is a primary amine which is cyclohexylamine, 2-ethylhexylamine, benzylamine, α-methylbenzylamine and tert-octylamine.

6. A salt as claimed in claim 1 wherein B is a secondary amine which is diethylamine, morpholine, dicyclohexylamine, N,N-methylbenzylamine or N,N′-dibenzylethylenediamine.

7. A salt as claimed in claim 6 wherein B is dicyclohexylamine.

8. A salt as claimed in claim 1 wherein B is a tertiary amine which is triethylamine, tributylamine, triisooctylamine, ethyldiisopropylamine, N-methylmorpholine, pyridine, 2,6-lutidine or quinoline.

9. A salt as claimed in any claim 1 wherein B is 1,1,3,3-tetramethylguanidine.

10. A salt as claimed in claim 1 wherein B is 1,5-diazabicyclo[4.3.0]non-5-ene (DBN) or 1,8-diazabicyclo[5.4.0]undec-7-ene (DBU).

11. A salt of formula (Ia)

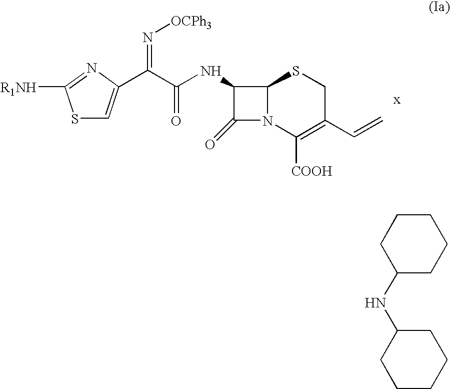

12. A method of preparation of cefdinir, comprising
reacting a compound of formula (II) 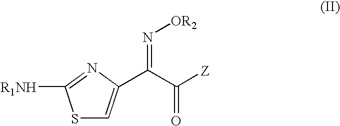
wherein
R₁ is hydrogen or an amino-protecting group;
R₂ is a hydroxy-protecting group; and
Z is a carboxy-activating group which is —Cl, —S-mercaptobenzothiazolyl, —O—P+(Ph)₃Cl—, —O—P(S)(OEt)₂, —O—P(O)(OEt)₂, —O—SO₂Me, —O—SO₂Ph, —O—SO₂-pTol, —O—COtBu, —O—C(O)OEt, —O-benzotriazol-1-yl, —S-(2-methyl-thiadiazol-5-yl), —O—CH═N⁺(CH₃)₂Cl⁻ or benzotriazol-1-yl-3-oxide,
with a compound of formula (III) 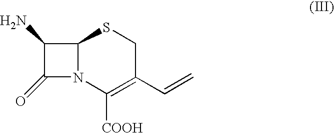
to produce cefdinir.

13. The method of preparing cefdinir of claim 12, comprising the steps of
(i) reacting the compound of formula (II) with the compound of formula (III) in the presence of a base, B; and
(ii) thereafter adding aqueous solvent and reducing the pH of the solution to obtain the acid of formula (V); 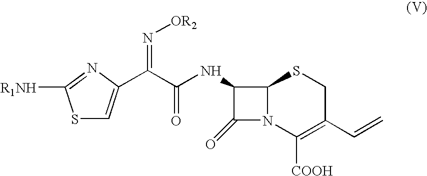
and
(iii) thereafter reacting the acid of formula (V) with a base, B¹, to cause precipitation of the salt of formula (I); 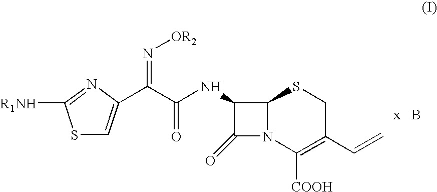
wherein
B and B¹ are independently ammonia or an organic base which is a primary amine, a secondary amine, a tertiary amine, guanidine, a guanidine derivative, or an amidine.

14. The method of preparing cefdinir of claim 12, comprising the steps of
(i) reacting the compound of formula (II) with the compound of formula (III) in the presence of a silylating agent which is N,O-bis-trimethylsilylacetamide, N-trimethylsilylacetamide, trimethylsilylchloride, hexamethyldisilazane, or trimethylsilyldimethylamine; and
(ii) thereafter adding aqueous solvent and reducing the pH of the solution to obtain the acid of formula (V); 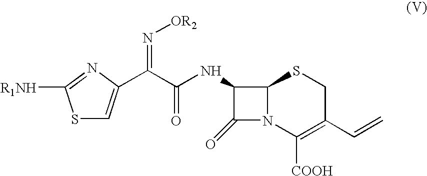
and
(iii) thereafter reacting the acid of formula (V) with a base, B, to cause precipitation of the salt of formula (I); 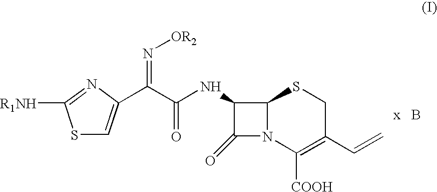
wherein B is ammonia or an organic base which is a primary amine, a secondary amine, a tertiary amine, guanidine, a guanidine derivative, or an amidine.

15. The method of claim 12, wherein
R1 is hydrogen, a C₁-C₆ acyl group optionally substituted with one or more fluorine or chlorine atoms, an alkyl- or aryl-oxycarbonyl group, or a trityl group wherein each benzene ring is optionally substituted with one or more methoxy and/or methyl groups; and
R2 is a straight or branched C₁-C₆ alkyl group, or a benzyl, benzhydryl or trityl group wherein each benzene ring is optionally substituted with one or more methoxy, nitro and/or methyl groups.

16. The method of claim 12, wherein
R1 is hydrogen or a formyl, tert-butoxycarbonyl, p-methoxybenzyloxycarbonyl, or trityl group; and
R2 is a tert-butyl, p-methoxybenzyl, 3,4-dimethoxybenzyl, benzhydryl, bis(p-methoxyphenyl)methyl or trityl group.

17. The method of claim 13, wherein
B and B¹ are independently cyclohexylamine, 2-ethylhexylamine, benzylamine, α-methylbenzylamine, tert-octylamine, diethylamine, morpholine, dicyclohexylamine, N,N-methylbenzylamine, N,N′-dibenzylethylenediamine, triethylamine, tributylamine, triisooctylamine, ethyldiisopropylamine, N-methylmorpholine, pyridine, 2,6-lutidine, quinoline, guanidine, 1,1,3,3-tetramethylguanidine, 1,5-diazabicyclo[4.3.0]non-5-ene (DBN) or 1,8-diazabicyclo[5.4.0]undec-7-ene (DBU).

18. The method of claim 14, wherein
B is cyclohexylamine, 2-ethylhexylamine, benzylamine, α-methylbenzylamine, tert-octylamine, diethylamine, morpholine, dicyclohexylamine, N,N-methylbenzylamine, N,N′-dibenzylethylenediamine, triethylamine, tributylamine, triisooctylamine, ethyldiisopropylamine, N-methylmorpholine, pyridine, 2,6-lutidine, quinoline, guanidine, 1,1,3,3-tetramethylguanidine, 1,5-diazabicyclo[4.3.0]non-5-ene (DBN) or 1,8-diazabicyclo[5.4.0]undec-7-ene (DBU).

19. An adduct of the salt of formula (Ia) of claim 11 with 2-mercaptobenzothiazole.

20. The adduct of claim 19 wherein the cefdinir:dicyclohexylamine:2-mercaptobenzothiazole ratio is about 1:1:0.5.
